(19) United States
(12) Patent Application Publication
(10) Pub. No.: US 2017/0127909 A1
(43) Pub. Date: May 11, 2017

(54) Apparatus for Clinical Use

(71) Applicant: Kenneth Francis Prendergast, London (GB)

(72) Inventor: Kenneth Francis Prendergast, London (GB)

(21) Appl. No.: 15/530,328

(22) PCT Filed: Jul. 2, 2015
(86) PCT No.: PCT/GB2015/000206

§ 371 Date: Dec. 27, 2016

(30) Foreign Application Priority Data
Jul. 7, 2014 (GB) 1412089.3
Jul. 11, 2014 (GB) 1412359.0
Feb. 19, 2015 (GB) 1502834.3
Jun. 29, 2015 (GB) 1511341.8

(51) Int. Cl. A61B 1/00; A61M 16/04; A61B 90/00; A61B 1/267; A61B 1/06; A61B 1/04

(52) U.S. Cl. CPC A61B 1/00048; A61B 1/00167; A61B 1/0684; A61B 1/04; A61B 1/00009; A61B 90/37; A61B 1/2673; A61M 16/0463; A61B 2090/372

(57) ABSTRACT

A apparatus adapted for use in clinically invasive proceedures comprising a plurality of objects forming a system, the object of which being to display to a surgeon or clinician the image of tissues and to improve the visualisation of tissue, and comprising: at least two computers or computer like devices containing microprocessors running independent operating systems; wherein, Component A, is any commercially available Tablet PC or smart phone or computer device capable of displaying high-definition video images having a touch screen capable of the pinch manouver; and is linked by BLUETOOTH® or equivalent to a second computer like device, Component B which has a separate microprocessor and operating system; and is linked using cables to a small sterile disposable component Component C which can attach to existing surgical retractors and comprises a LED (light emitting diode) or equivalent and a CMOS pixel sensor chip or equivalent.

The present disclosure claims priority from the folowing UK patent applications: GB1511341.8 filed on 29 Jun. 2015; GB1502834,3 filed on 19 Feb. 2015; GB1412359.0 filed on 11 Jul. 2014; and GB1412089.3 filed on 7 Jul. 2014.

### TECHNICAL FIELD

The invention discloses an apparatus adapted for use in invasive clinical procedures comprising a plurality of devices. In particular, the present apparatus forms a system from a plurality of objects working in concert comprising: computers; novel devices with lighting and camera image sensor components; attachments; connecting and covering devices working together as a system whose object is to improve visualisation during surgery. More particularly the invention also discloses novel devices useful in the performance of surgery and other invasive clinical procedures.

### BACKGROUND ART

The field of surgery is a difficult art to practice. A generally held misconception is that surgery requires particularly high levels of manual dexterity. This is not the case. In fact the level of manual dexterity required for surgery is probably below that required for elementary skill playing a musical instrument. Despite the relatively simple nature of surgical tasks however, surgery is difficult. The factors which make surgery difficult lie in a second common misconception. In particular, many people are shocked to learn that surgeons often cannot clearly see those tissues and vital structures being operated on in the depths of a wound. The great unrecognised problem in surgery is the fact that surgeons cannot see what they are doing, especially when they need to do so most.

Although surgeons can read a newspaper using a standard desk lamp, in operating theatres however, surgeons make use of large circular arrangements of powerful light sources. The fact that arrays of powerful light sources are used in operating theatres leads many to assume that seeing structures in a wound should not be a problem for surgeons. In fact, the need for such powerful light sources should be an indication that seeing structures deep in a wound is a major unresolved problem. Surgery becomes difficult when clear visualisation of structures cannot be achieved. Further, a primary reason for surgical error lies in poor visualisation of anatomical structures. Poor visualisation increases operating time and increases the risks.

Thus the present disclosure teaches away from commonly held beliefs and teaches a means for assisting surgeons to better see what they are doing and teaches that surgery would be easier to perform and safer if surgeons could see what they are doing more reliably.

It is an object of the present disclosure to provide an apparatus adapted for use in clinically invasive proceedures comprising a plurality of components which when working together form a system which makes it possible to harness the powerful image presenting capabilities of Tablet PC's or smart-phones or advanced goggles and thereby to improve the reliability of visibility during surgical operations.

At the time of filing it is the practice in the art of surgery that for some procedures surgeons obtain improved visual access or magnified views of deep body spaces such as; the middle ear, spaces in the head and neck, the hylum of the liver, vocal chords; oesophagus abdominal cavity and the like using either fibre-optic cable endoscopes or operating microscopes.

Fiberoptic endoscope devices are usually separately lit up by means of an external lighting unit which is heavy and expensive and non disposable. The non disposable fiberoptic cables are additionally expensive to clean and maintain. Moreover, the cables themselves are easily broken by twisting thus degrading image quality over time with repeated use. Typically, the surgeon looks through the fibre-optic endoscope using an eyepiece. However when looking through the eyepiece the surgeon's view is restricted only to that view obtained from the eyepiece. Thus making it necessary to put down the fiberoptic device if the surgeon wishes to look directly at the wound. Thus it is not easy for surgeons to switch between direct visualisation of a wound and the fiberoptic cable endoscope view during procedures. Fiberoptic endoscopes are also used to inspect cavities such as the colon. The adapted apparatus of the present disclosure may be used in a similar manner.

In the case of operating microscopes these are heavy pieces of equipment requiring the surgeon to remain seated and position the large operating microscope lens and eyepiece unit directly in front of the wound so as to look through the lens eyepiece. The size of the lens and eyepiece and its position makes it impossible to see around it. Further, it is time consuming to position the lens correctly. Thus alternating between a direct view of the wound to a magnified views is not easy. Further, operating using microscopes makes it inconvenient to change instruments or pass instruments from scrub nurse to surgeon. Further still, it is difficult to pass instruments between scrub nurse and surgeon when using such microscopes without the risk that the heavy lens unit might be knocked and thereby moved out of focus; thus wasting surgical time continually adjusting and repositioning the said microscope. Operating microscopes are additionally expensive and heavy pieces of equipment difficult to clean and maintain.

Operating microscopes find use during hand surgery and during anastomosis of nerves or vessels, micro-surgery, during which procedures surgeons would appreciate the flexibility of being able to switch easily between a direct view of the wound and a magnified view.

It is an object of the present disclosure to seek to overcome some of these problems.

### BRIEF DESCRIPTION OF THE INVENTION

The present disclosure provides a novel apparatus comprising a plurality of components some of which are non-disposable and some disposable forming a system whereby the surgeon obtains an improved view of the depths of the wound displayed on a Tablet PC preferably, or equivalent device, Component A. It will be appreciated that Component A could be a Tablet PC a laptop computer or other computer device or equivalent device with a screen such as a smart phone, or advanced goggles such as Google Glass or equivalent device.

The Tablet PC providing the view of the operating field to the surgeon may held in front of the surgeon by a scrub nurse or placed in a convenient position mounted on a suitable stand adapted for the purpose. This way, by means of the novel apparatus the surgeon can alternate his view from a direct view looking down into the wound or he may easily switch to an indirect view obtained by looking directly at screen of the Tablet PC.

Moreover, the image displayed on the said preferred Tablet PC may be easily magnified by pinching the screen using the pinch manoeuvre, which could be carried out either by the surgeon if the screen is rendered sterile, or covered by a suitable sterile membrane, or by a scrub nurse or by a runner or by an ODA (Operation Department Assistant).

It is further envisaged that when in use in operating theatres the Tablet PC would be placed on a suitable stand and that the said Tablet PC and some or all of the stand should be covered by a clear transparent preferrably sterile plastic disposable membrane so as to prevent the said Tablet PC from being contaminated by spatter from contaminated fluids and so as to prevent contaminants on the non-sterile Tablet PC from contaminating the scrubbed personnel.

The resultant images and improved lighting in the depths of the wound are obtained as a result of the novel synergistic system of components working together forming the apparatus which would typically include additionally a small disposable component, Component C, described more fully in the embodiments and disclosed herein below. This small disposable component, Component C, for the purpose of lighting and image sensing is linked preferably by electromagnetic conductive cable or by other means disclosed hereunder to a separate intermediate mini-computer, computer system, Component B, which Component B may take the form of a dongle.

This intermediate computer or computer like device, Component B, pulls an image data stream from Component C, the disposable component and pushes the image data stream to Component A for display. Component B could be any computer device even one similar to that of Component A. However, Component B would preferably and more typically take the form of a dongle like device specifically configured for its restricted purpose and comprise a minimum of components such as some or all of the following:

a CPU microprocessor; a buffer RAM; buffer cache; read only memory with the operating system which would be a separate operating system functioning independently from that operating system running on Component A, the Tablet PC or smart phone or equivalent. Moreover the operating system of Component B may be similar to that of Component A but is envisaged to be different and may be a slimmed down kernel operating system selected from the many available.

Additionally Component B comprises:

BLUETOOTH® chip components; USB port with associated chips and other sub components disclosed here below. Thus the image data is pulled by the intermediate computer, Component B, from Component C and pushed by Component B, by BLUETOOTH® preferably, to the Tablet PC, Component A. The present disclosure provides the various components and the orchestration of the same which are orchestrated to work together as an ensemble.

The small image gathering (pixel sensor) and light source component, Component C used in the depths of the surgical wound is envisaged to be disposable, cheap and sterile. The present disclosure provides an apparatus adapted for use in invasive clinical procedures comprising a plurality of components which are linked together to form an integrated system with the Tablet PC, Component A. The present disclosure also provides novel components such as the disposable lighting and image gathering device, Component C, and which disposable Component C may be preferably attached by suitable attachments to pre existing surgical retractors in current use.

It will be further appreciated that Components B and C may be combined or fused together to form a lighting and image gathering device which is also computer like, dongle like, combining the features of the computer Component B with those of Component C, as disclosed in a further exemplary embodiment.

This novel lighting and visualisation system as disclosed herein is not limited to surgical procedures and also has applications in the field of anaesthesiology to permit visualisation of the vocal cords during difficult intubation and other clinical and diagnostic procedures.

It will also be appreciated that the disposable Component C may be packaged and given to the patients after first use and the patient instructed to bring his/her own Component C to review clinics and the like. This would be of particular benefit for head and neck cancer patients who need regular reviews and examination of the back of the throat for many years following their surgical treatment.

### BRIEF DESCRIPTION OF THE DRAWINGS

The disclosure is more fully illustrated by reference to the diagrams, not drawn to scale, which are intended to be illustrative and to the exemplary embodiments set forth here below. The invention will now be further illustrated by reference to the Figures which are not to scale and intended to be illustrative.

FIG. 1 (a) depicts an overview of the invention in use exemplified by one particular example of use and one particular embodiment. FIG. 1(a) depicts a Tablet PC, Component A, possibly hand-held, but in this instance supported on a stand appropriate for use in an operating theatre. The design of the stand is trivial and not specified in the present disclosure apart from the specification that the stand should be manufactured from materials which enable it to be rendered sufficiently clean by suitable disinfectants and cleaning agents appropriate for use in operating theatres. The stand holds the Tablet PC in a manner so as to permit the surgeon to view the images displayed on it.

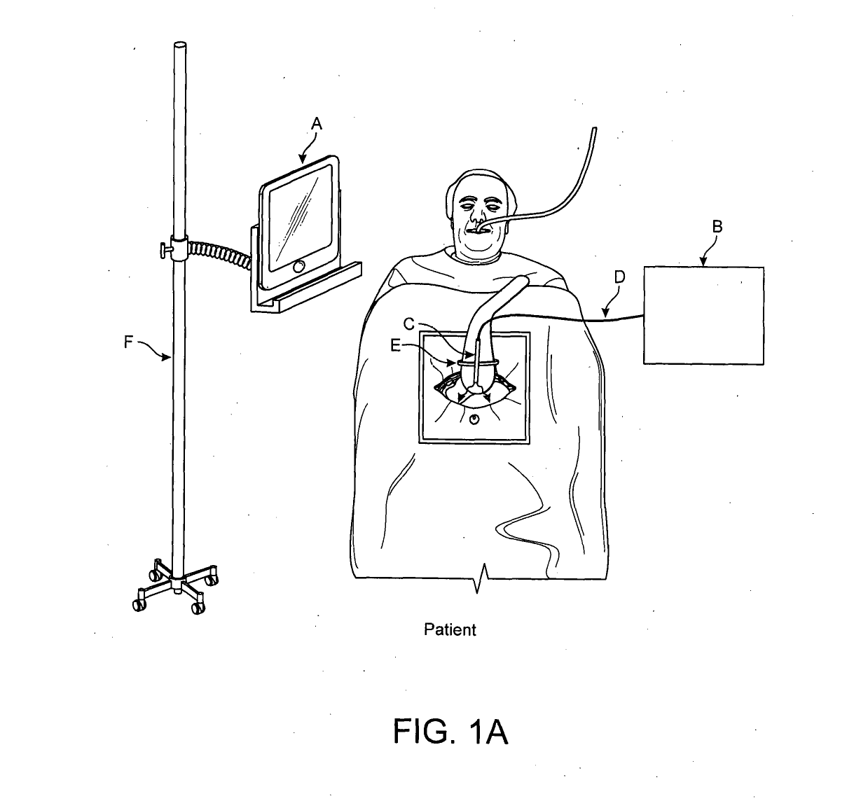

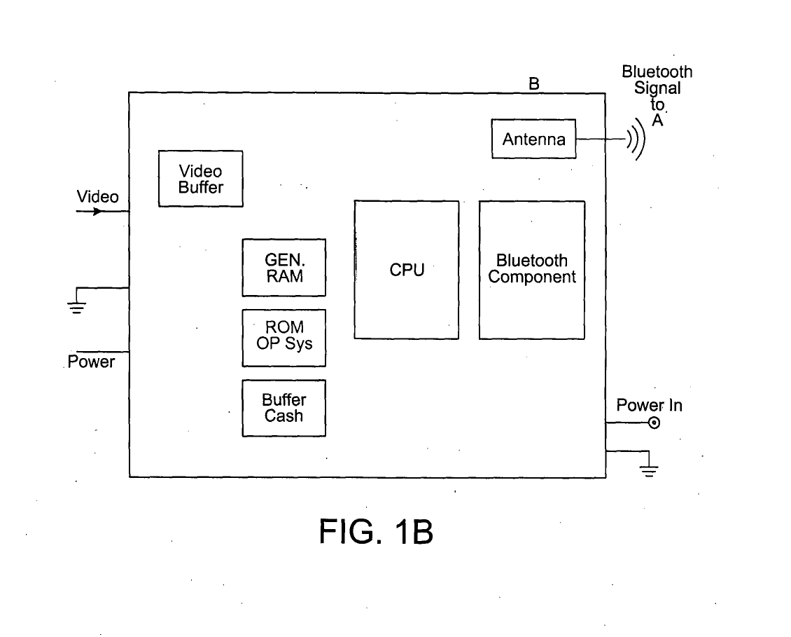

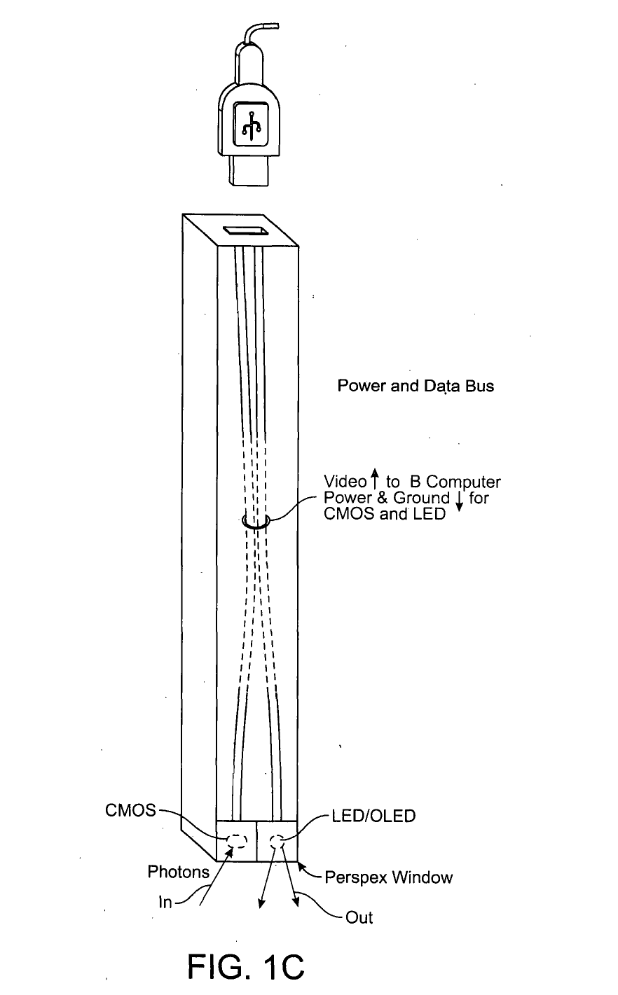

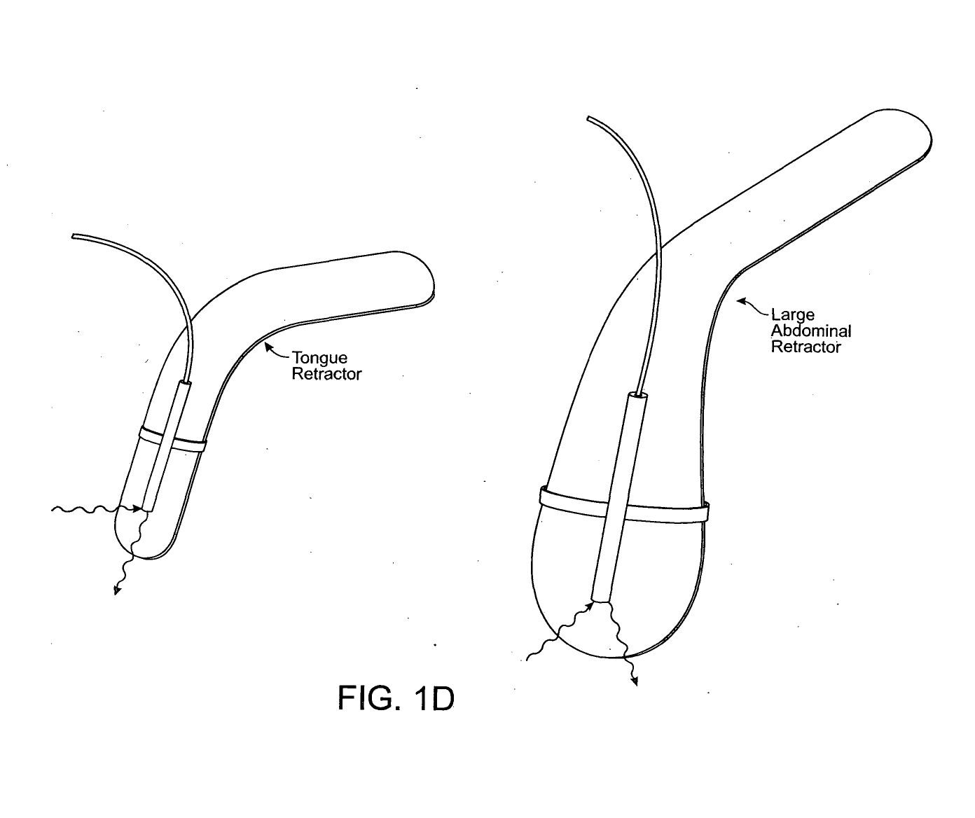

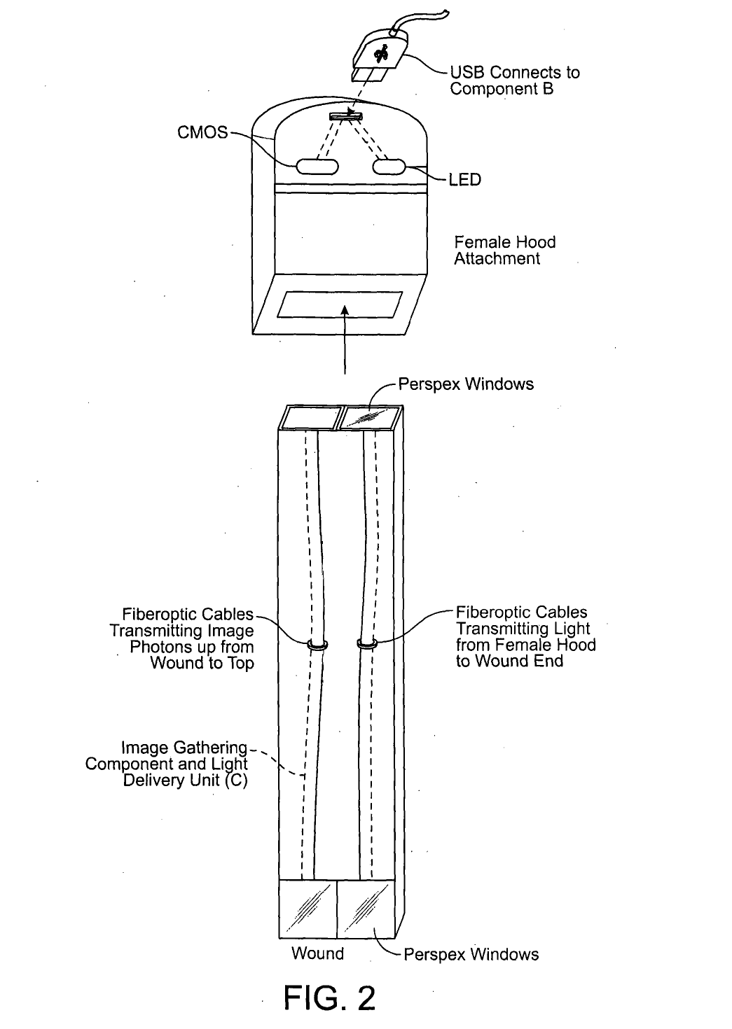

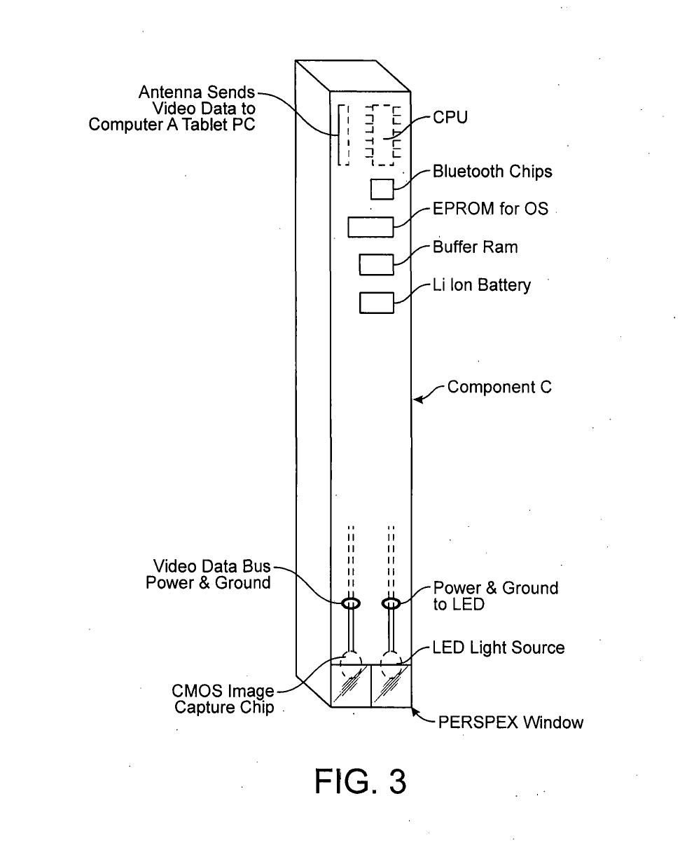

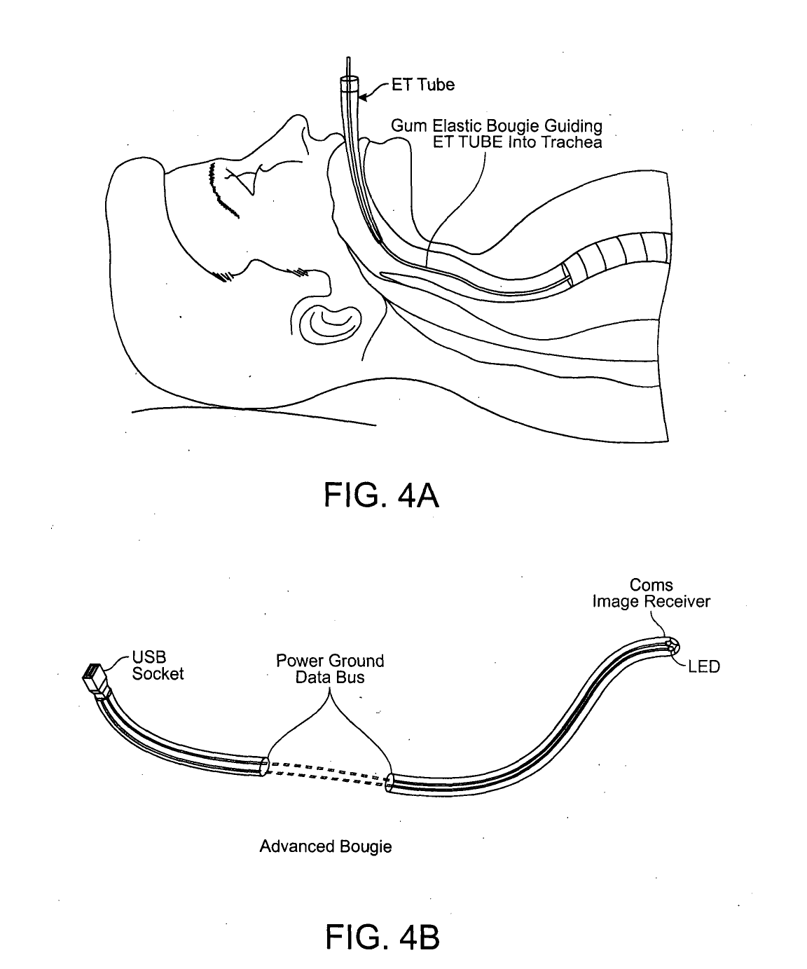

In this diagram a Tablet PC only is displayed. However it will be appreciated that Component A could also be a smart-phone, laptop, desk top PC, or net book. It will be further appreciated that Component A could take the form of computerised glasses such as Google Glass® or equivalent. The preferred Tablet PC, Component A of the synergistic system, displays images from the wound to the surgeon. The diagram FIG. 1(a) depicts a patient lying on the operating table undergoing abdominal surgery. A second small dongle like computer, Component B of the system, having its own independent operating system which separate operating system running on component B may be, but is not limited to, any of the following operating systems: Android One®; Android operating systems 4.4 or earlier or later versions such as Ice Cream Sandwich® or Android Jelly Bean® or Android Kit Kat®; Contiki®; Java operating systems; Java based operating systems for embedded devices; Microsoft Internet of Things; Microsoft Windows 8® or earlier or later versions thereof; Microsoft Windows Phone 7.5 or earlier or later versions thereof; Linux or versions thereof or or slimmed down version of the Linux kernel; Apple iOS7® or earlier or later versions thereof to mention some but by no means all of the operating systems which could be run on Component B.

Component B transmits a video data stream by BLUETOOTH® signals to Component A, the Tablet PC, which received data signals from Component B are processed into a visual image which is visualised by the surgeon the screen of Component A. Component C of the system is the disposable light emitting and image collecting component which is depicted in this case attached by an unspecified and trivial attachment device to a standard large abdominal retractor which in this case is holding back the liver. Component C, the sterile disposable component generates a video image data stream which is pulled by Component B which in turn pushes the data stream to Component A.

FIG. 1 (b) depicts a schematic diagram for the mini computer, dongle like, Component B of the system which has its own operating system. The mini computer Component B receives video image data stream and supplies power and ground ground to the disposable image gathering, pixel sensing, and light delivery component, Component C of the adapted apparatus forming the system.

The mini computer Component B will typically comprise some or all of the following;

a CPU; a RAM video buffer; general RAM; an EPROM (Erasable Programmable Read Only memory) or ROM (Read only Memory) or PROM (Programmable Read Only Memory) chip; providing an operating system, which operating system would be typically a slimmed down kernel; additional optional buffer cache where desired; Bluetooth® circuits and an antenna. The Bluetooth® chip component and antenna transmits the video image data stream to Component A, the Tablet PC or smart phone.

FIG. 1 (c) depicts a schematic illustration of the disposable image gathering and light delivery component, Component C, of the synergistic system, composition of objects. The image gathering and light delivery component, Component C of the synergistic system is depicted as a oblong object which is rectangular but could be cuboid or indeed any desired shape. However an elongated shape is preferred, so as to extend the image gathering and light delivery component, Component C of the apparatus out of the surgical field at the top end so as to permit the attachment of a USB cable to a suitable attachment at the top end of Component C some distance from the depth of the surgical wound, to avoid compromising sterility requirements.

It is envisaged that the image gathering and light delivery component, Component C of the apparatus itself comprises some or all of the following sub-components:

an outer skeleton which would be disposable sterilizable hard plastic which may be rectangular/cuboid or cylindrical. The plastic case could be manufactured from Nylon or Perspex or Polyethylene or the like;

in the region most inferior of Component C, that part located closest to the depth of the wound there would be typically be located behind one or more Perspex window(s) a CMOS device (video camera chip) pixel sensor which receives light photons from the wound and generates a video image data stream.

The said Perspex window(s) may be shaped so as to serve the additional functionality of an appropriate lens. Which lens functionality would emit and receive light photons from the desired direction which could be along the long axis of the Component C or tangential to its long axis or at an angle to it with the appropriate focal length configured as desired. Alternatively in front or behind the Perspex window a separate lens specie may be provided.

alongside the CMOS image sensor chip or close by lies an LED (light emitting diode) or OLED (Organic Light Emitting Diode) or equivalent specie which emits photons to illuminate the surgical field through the same or through a separate Perspex window. The window is preferably manufactured from Perspex but may be manufactured from other suitable materials such as glass and the like,

the light emitting diode or other similar similar light source is powered and grounded by means of electro conductive cables included in a data bus which is included within Component C and traverses the length of the image sensing and light delivery component, Component C of the apparatus forming the system,

the data bus or equivalent electromagnetic conductive cabling additionally conveys Video data signals from the CMOS pixel sensor chip component upwards towards a USB attachment and conveys power and ground down to power the CMOS and LED components respectively,

typically a USB cable or equivalent is inserted into a suitable male or female attachment located at the top of the image gathering light delivery component, Component C, of the apparatus forming the system.

FIG. 1(d) depicts two surgical retractors to which an exemplary embodiment of Component C is depicted as attached. A tongue retractor is depicted which has a sterilized image gathering and light delivery component, Component C of the apparatus attached to the said tongue retractor by a separately adapted attachment specie, such as a clip or clasp or other suitable attachment mechanism of attachment the nature of which is trivial and readily apparent to the skilled artisan and will not be specified here in the disclosure, in the interests of brevity.

Also depicted is a large abdominal retractor with a schematic depiction of a clip or attachment device attaching the image gathering and light delivery component, Component C of the adapted apparatus to that abdominal retractor.

It can be seen from the diagram that a USB cable attaches to the top of this image gathering and light delivery component, Component C of the apparatus forming the system.

Surgeons are by and large conservative and prefer using the equipment they have been used to using. Thus it is an object of the present disclosure to provide digital hardware components which can work in tandem with existing surgical retractors and the like which surgeons are familiar with.

Accordingly, Component C is envisaged to have a disposable pen like shape and configuration and similar dimensions to facilitate the attachment of said Component C to pre-existing surgical retractors in current or future use by suitable attachment specie and mechanisms which attachment specie are trivial and readily apparent.

FIG. 2.

FIG. 2 depicts a cheaper embodiment of the image gathering and light delivery component, Component C. This variation provides a means of preserving the CMOS image sensor and LED sub-components to avoid disposing of these. In this embodiment the CMOS image sensor and LED sub-components are not present in Component C but placed instead in an intermediate device illustrated in this instance, but not limited to being, a detachable hood type configuration. FIG. 2 depicts a separate detachable female hood attachment which is capable of connecting to a USB cable or similar cable which USB cable would attach the intermediate computer unit, dongle like, Component B, to the hood. This USB connection would provide power ground control and data bus services to the CMOS image sensor sub-component and LED sub-components located in the hood.

The female end of the hood would typically comprise, but not necessarily be limited to comprising, a suitably shaped opening here depicted as a rectangular shaped opening into which the top of Component C would be inserted.

Image gathering and the light delivery would take place through the top of Component C where at the top of Component C a Perspex window would make intimate contact with a similar Perspex window just beneath the CMOS image sensor and LED sub-components.

Strands of fibre-optic cables running from the top end of the image gathering light delivery Component C to the bottom end of Component C, close to the wound, would respectively convey light photons down so as to illuminate the wound and convey reflected image photons up to the CMOS image sensor chip located in the hood. The strands of fiber-optic glass cables would typically terminate at appropriate Perspex window(s) and emit and collect light through the said Perspex window(s) which window(s) could be configured to serve the function of a lens as described herein before.

In use the detachable head containing the CMOS chip and LED components could be covered and isolated by suitable disposable plastic cover sheaths thereby permitting the hood to be suitable cleaned and rendered fit for reuse after the operative procedure.

The remainder of Component C, the pen shaped most inferior Component C which had been attached to the hood and which was located in the depth of the wound could be simply disposed of and incinerated. Thus avoiding the need to dispose of the expensive CMOS and LED subcomponents located in the hood and thereby reducing environmental waste.

FIG. 3 depicts a more expensive version of Component C of the synergistic light emitting image collecting system for use in clinically invasive procedures and surgery.

In this embodiment Component C comprises its usual components combined additionally with those components normally present in Component B, the intermediate computer component, dongle.

In particular, this expensive embodiment of Component C comprises some or all of the following:

a Perspex window at the wound end, with the CMOS image capture chip behind one Perspex window; linked by

a video data bus; conveying a data stream upwards to

the CPU processing chips all contained in the same sterile disposable device. The bus provides power and ground and transmits video data upwards by the same bus, alongside the CMOS circuit or close by lies an

LED or appropriate light source; similarly powered and grounded by the data bus; and other associated cables which serves both the CMOS and light emitter component; and additionally those components normally present in Component B comprising some or all of:

a CPU chip;

BLUETOOTH® chip(s) circuits or equivalent;

the EPROM chip or ROM or Ram or Flash or equivalent memory storage device holding the independent operating system;

RAM buffer cache(s); and

lithium ion battery for power or other power source, which power source may also be externally supplied by a jack, not depicted in the schematic illustration.

The various chips contained in Embodiment 3 of Component C of the adapted apparatus forming the system will be linked together by appropriate wiring, not shown, and or by a suitable circuit board, unspecified, which wiring diagram will be configured to take account of the final desired shape and layout and which wiring layout or circuit board being a matter trivial to the skilled artisan, skilled in the art of laying out wiring diagrams and circuit boards for electronic devices. The various chips of Embodiment 3 of Component C of the apparatus will ultimately be connected to an antenna which will transmit the video image data stream to Component A, the tablet PC of the system not shown in this particular diagram, the preferred Tablet PC, or equivalent.

FIG. 4

FIG. 4 (a) illustrates the basic problem of difficult intubation where a gum elastic boogie is depicted guiding an endotracheal tube (ET tube) past the vocal cords into the trachea.

FIG. 4(b) depicts a modified embodiment of Component C which has been elongated to take the form of a typical gum elastic bougie used in anaesthetics and which incorporates and comprises those electronic sub-components normally found in Component C described herein above and which functions in a manner in accordance with Component C as described herein above. Thus permitting a computer rendered view of the vocal cords on a suitable Component A, Tablet PC, smart phone, or the like enabling an anaesthetist to visualise the vocal chords on the Tablet PC or convenient smart-phone.

### DETAILED DESCRIPTION BEST MODE AND EXEMPLARY EMBODIMENTS

The disclosure will be further explained by reference to the exemplary embodiments set forth here below;

### Embodiment 1

Attention is drawn to FIG. 1A which depicts the invention in use in general terms with reference to one particular mode of use and one particular embodiment.

Figure 1A depicts a Tablet PC, normally hand-held, but in this instance supported on a stand, suitable for use in an operating theatre, which stand holds the Tablet PC in a suitable manner to permit the surgeon to view images displayed on it.

The hand-held PC tablet PC or equivalent, Component A of the system, displays images from the wound to the surgeon.

The diagram FIG. 1 (a) depicts a patient lying on the operating table undergoing abdominal surgery.

The apparatus comprising a plurality of objects adapted for use in clinically invasive procedures is depicted comprising various components in particular;

a second small computer or dongle, Component B of the system having its own operating system, which Component B transmits video image data via BLUETOOTH® signals or equivalent to the Tablet PC, Component A.

The said video data signals are then rendered into a visual image of the wound by Component A which images are then viewed by the surgeon.

Component C of the apparatus is the disposable light emitting and image sensing component which is depicted in this diagram attached to a large abdominal retractor. The abdominal retractor, in this case, is depicted as holding back the liver.

In the depths of the abdominal wound the sterile disposable Component C comprises:

a light source possibly but not limited to a light emitting diode, which emits light at the base of the Component C through a suitable window made of Perspex but not limited to Perspex;

alternatively fibre-optic cables could transmit light down through the structure of Component C;

a CMOS image sensor or Charge Coupled device chip circuit video camera device located at at the base of component C, or any similar component having the properties of a CMOS video camera namely the capability of converting reflected photons into a digital data stream suitable for image processing, which CMOS sub component detects reflected light photons from the wound;
a data bus traversing the length of Component C which could be attachable to a cable, which preferred cable could be, but is not limited to being, a USB cable and this cable may be disposed of after the surgical procedure or may be reused; in which case the cable would covered during the surgical procedure by a suitable sterile disposable plastic sheath.

The captured image data is then conveyed to the intermediate computer, dongle Component B, by means of the attached USB cable.

It is envisaged that the dongle mini computer Component B would in use be covered by suitable sterile, disposable, fluid impermeable, surgical drapes to minimise contamination of its outer surface by spatter with body fluids. It is further envisaged that the dongle being preferably non-disposable should be manufactured with an outer shell or casing capable of withstanding regular cleaning by those cleaning agents appropriate for use in surgical operating theatres. Additionally the surface of Component B, the dongle, should have the minimum number of crevices cracks or spaces thereby minimising the risk of the dongle, Component B, harbouring colonies of bacteria or pathogens and becoming a source of infection.

Component C, the disposable light emitting and image collecting component is preferably attached to the preferred surgical retractor by means of a suitable attachment specie. Alternatively Component C may be held by an assistant in the wound.

Each the these components Components A, B, C, respectively will be discussed in further detail here below.

It will be appreciated that Component A, preferably a Tablet PC or smart-phone, or advanced computerised goggles could be but is not limited to being one of the following: an Apple iPad®; iPad mini®; Amazon Kindle Fire HDX7®; Samsung Galaxy Note Pro 12.2®; Microsoft Surface Pro 3®; LG G Pad®; Samsung Galaxy Tab Pro10.L®; Google Nexus®; Sony Xperia®; HTC One®; Nokia Lumia and the like or Google Glass® and the like or earlier or later versions of the above. It is envisaged that the Tablet PC or smart phone will be capable of displaying high-quality images and receiving and displaying the video image data. Data would typically be received by Component A, the Tablet PC by means of BLUETOOTH® signals or equivalent, as the preferred means of pushing the said video image data stream from Component B to Component A. Although it will be appreciated that the video image data stream may be conveyed to Component A, the Tablet PC, by means of a wire connection if so desired, although this is not preferred, without departing from the scope and spirit of the invention.

The present disclosure teaches that data transmission via BLUETOOTH® is the preferred method for conveying the image video data stream from Component B to Component A for final rendering and display of images. Transmission of data by Bluetooth® or equivalent electromagnetic radiation transmission is preferred over transfer of data by wire cable between Component B and A for a number of reasons particular to the art of surgery.

First it is a teaching of the present disclosure that any wire physically connecting Component A the Tablet PC which will display the images, to Component B or a combined embodiment combining Components B and C or directly to Component C, could transmit physical forces from Component A down to the depth of the wound, if Component A were unexpectedly moved about. Such transmitted forces could disturb the retractor or even lead to Component C moving about unpredictably in the depths of the wound and possibly bring about damage to delicate vital structures.

It is a further teaching of the present disclosure that there should be no direct physical link between Component C which lies in the depth of the wound and which would be potentially exposed to transmissible infectious agents such as HIV, Hepatitis B or C viruses or other infectious agents and which of necessity requires safe disposal of Component C. Thus if there were a direct physical wlre connection between Component C and Component A this could lead to a legal requirement to dispose of the expensive Tablet PC, Component A. Accordingly taking account of the particular needs of the surgical environment the present disclosure teaches the best mode to achieve the objects of the present disclosure. Which objects are in particular:

improving the illumination of structures in the depths of a wound;

capturing an image of structures of interest in the said wound;

displaying an image of the said structures to the operating surgeon; and in a manner without risking undue damage to the said vital structures; and without increasing the risks of contamination by infectious agents; and at the same time, permitting the surgeon to view the anatomical structures by easily switching between views magnified and un-magnified direct and indirect separately or viewed in combination; and where the displayed image may be moved about in three dimensional space and is not fixed in a position directly in front of the wound and thus not obscuring the surgeons direct view of the wound.

The present disclosure teaches that the best mode to achieve these objectives and envisages three primary components, Components A, B, C acting in concert as a synergistic system comprising a plurality of objects. In particular; Component C which lies in the depth of the wound and is disposable and which is connected by wire to Component B, a dongle like mini-computer device which may be covered by plastic sheaths and or sterile drapes and which Component B has no physical connection to Component A which Component A must be free of any direct physical connection to the surgical wound.

Thus the apparatus forming the system of necessity requires that Component B, the dongle like mini computer must have its own independent operating system separate from that of Component A.

The Tablet PC, Component A, will additionally preferably have a touch sensitive screen and be capable of displaying a magnified version of the image. Thereby providing the surgeon with an easily accessible source of magnified imaging of the depths of the wound and the vital structures therein. In use the Tablet PC Component A may be covered by a sterile transparent plastic film or cover sleeve to prevent contamination of scrubbed personnel by the said tablet PC.

This teaching of the present disclosure provides a further object of the invention in particular a more user friendly means for surgeons to view magnified views of anatomical structures which additionally permits the surgeon to rapidly change the degree of magnification and which permits the surgeon to alternate conveniently between a direct vision view of the wound and an indirect view displayed on the Tablet PC, Component A and view a magnified indirect view and a direct unmagnified view of the wound simultaneously if so desired.

Thus the present disclosure teaches away from existing surgical microscopes currently used in surgical operating theatres at the time of filing, which existing surgical microscopes require the large lens and eyepiece units to be precisely focused and held in one constant position usually directly in front of the wound, hence the need for heavy mountings and support structures for existing surgical microscopes. The large lenses and eyepieces of existing surgical microscopes situated directly in front of the wound also prevent the surgeon from seeing around them making a direct un-magnified view of the wound difficult. Moreover, in use the focus of existing large surgical microscopes is often lost if the the large unwieldy microscopes are moved or knocked by assistants or other operating theatre personnel.

The adapted apparatus forming the system taught by the present disclosure forming an apparatus adapted for use in invasive clinical procedures disclose a means for displaying an image, magnified or un-magnified of anatomical structures but where the display is not necessarily fixed in one position in three dimensional space. More particurlarly the display is not restricted to being directly in front of the wound, but which display can be positioned anywhere close to or at a distance from the surgeon without causing loss of focus. Because the display mechanism Component A of the system, the Tablet PC, is not physically connected to the other components of the system, Components B, and C. Further because Component A preferably receives its image data stream input by means of WiFi or BLUETOOTH® or equivalent this makes it possible for Component A to be placed in any convenient position relative to the surgeon thus permitting even a simultaneous direct view of the wound and simultaneously an indirect view magnified or unmagnified if so desired.

Attention is further directed to FIG. 1(b) which depicts a typical schematic diagram for the intermediate computer Component B, the dongle component of the apparatus which runs its own operating system.

The intermediate computer, Component B, receives a video image data stream from and supplies power and ground to the disposable image gathering and light delivery component, Component C of the adapted apparatus preferably via USB connections and cable(s) or functionally equivalent cables and connections.

The intermediate mini computer dongle, Component B would typically comprise some or all of the following sub-components:

a CPU;

a RAM buffer for the video image data stream;

general RAM for the operating system;

an EPROM (Erasable Programmable -Read Only memory) PROM or a ROM or Flash memory chip or equivalent for storing and providing an operating system, which operating system could be typically a slimmed down operating system kernel or a full operating system if reduction of code is not desired;

additional optional buffer cache memory where desired;

BLUETOOTH® circuits and an antenna; which BLUETOOTH® chip component and antenna transmits the video image data stream to the Tablet PC;

and switches and indicator lights and appropriate circuit boards and power sources and the like where desired which are not shown in the schematic diagrams, in the interests of brevity, but which switches and indicator lights are trivial and readily apparent to the skilled artisan in the field of electronics;

an outer skeleton or casing manufactured from material capable of withstanding cleaning by those agents commonly employed in surgical operating theatres.

The intermediate computer Component B, the dongle would normally receive power and ground by battery or external jack connections to suitable power sources. Component B in turn provides power and ground to Component C. Plastic objects such as Component C which have had contact with body fluids during surgery require careful disposal to avoid the spread of infection. The safe disposal of objects contaminated by body fluids is expensive and normally takes place by high temperature incineration. Thus the sterile disposable Component C should preferably be manufactured from materials readily degradable by incineration and should leave the minimum of post incineration residue. Moreover it is also preferable that the smoke emissions from the incineration of Component C should contain the least amount of heavy metal vapours as far as practicable.

Accordingly it is an object of the present disclosure to modularise the system of components into three principle components. Components A, B, and C where the sterile disposable Component C has a minimum amount of heavy metal containing sub-components so as to facilitate disposal.

In one embodiment depicted in FIG. 1 C the image gathering and light delivery component, Component C of the apparatus is depicted as a oblong object of rectangular or cuboid shape It will be appreciated that Component C could be cuboid or cylindrical or cigar shaped L-shaped, cuboid, ellipsoid or any suitable shape without departing from the scope and spirit of the disclosure.

However preferably, surgeons would prefer the image gathering and light emitting Component C to be of an elongated shape so that the USB cable may be attached to the top of Component C, of the system, some distance away from the wound.

It will be appreciated that a narrow elongated pen shape is also preferred for the additional reason that a pen shaped Component C would obscure the surgeons view of the wound less than other shapes and additionally be less in the way of surgical instruments.

Typically, Component of C is envisaged to be, but not limited to being, a long rectangular unit measuring 1 cm deep 1 cm wide and perhaps 10 cm in length. Another preferred shape for Component C is cylindrical, having measurements largely similar to those of a disposable pen. The dimensions of the pen shaped variation of the embodiment are envisaged to be, but are by no means limited to being, a radius of 3 mm and length 10 cm. This circumference may be expanded at the top of the device to accommodate a suitable USB or equivalent attachment. It will be apparent however that many variations in the dimensions of the form of this embodiment would be readily apparent to the skilled artisan without departing from the scope and spirit of the invention as disclosed.

Further still, an elongated shape is preferred so as to extend the image gathering and light delivery component, Component C of the synergistic system out of the surgical field so as to permit the attachment of USB attachment some distance from the depth of the surgical wound, which wound must remain sterile.

Thus the image gathering and light delivery component, Component C of the synergistic system would typically comprise the following sub components:

In the region most inferior which would be located in the depths of the wound there would typically be located behind a Perspex window a CMOS subcomponent, video-camera chip with appropriate circuit, which receives reflected light photons from the wound and converts the said reflected light photons into a digital data stream of video images. The Perspex window could be configured to serve the functionality of a lens as described herein before.

Alongside the CMOS chip or close by lies an LED (light emitting diode) or equivalent light source emitting photons through a separate Perspex window or the same window configured such that the photons emitted from the LED do not interfere with the reflected photons from the wound.

The light emitting diode or other similar similar light source is powered and grounded by a electro conductive cables or wires which may be arranged as a data bus which traverses the length of the image gathering and light delivery component, Component C of the synergistic system. The bus or cables also conveys a video data stream from the CMOS chip circuit upwards towards the USB attachment and conveys power and ground down to power the CMOS and LED components respectively.

The USB attachment is typically inserted into the top of the image gathering light delivery component, Component C, of the synergistic system.

The said USB cable or equivalent cable may be disposed of after surgery together with Component C or may be covered in use by a disposable sterile plastic sheet or sleeve. Not shown in the diagram is the presence of an optional flange around the top of the light emitting video image sensing disposable Component C to permit the attachment of such a sterile disposable plastic cover sheet or sleeve.

It will be further appreciated that the USB cable attachment could take place some distance from the top of Component C in which case a cable would emerge from the top of Component C leading to a USB attachment. This short length of cable could be disposed of with Component C as this short cable would be physically attached to Component C. Such a configuration would prevent a need to dispose of a full length of USB cable.

It is further envisaged that the sterile disposable Component C will come packaged in double layered packaging where the outermost surface of the layer of the outer packaging is non sterile but where the inside surface of the outer layer is sterile and the inside layer is sterile on both surfaces of the inside wrapping.

Attention is further directed to FIG. 1(d), which is not drawn to scale, which depicts two surgical retractors. On the left, a tongue retractor which has the image gathering and light delivery component, Component C of the apparatus attached to the said tongue retractor by a separately adapted suitable attachment.

It will be noted that surgeons prefer to use those retractors they are familiar with and thus the image gathering and disposable light delivery component, Component C of the apparatus forming the system may include in its superstructure or may be provided with a separate suitable attachment mechanism capable of attaching the said Component C to whatever traditional retractor a surgeon prefers to use. Such attachment mechanisms are not specified as there are many suitable attachment mechanisms available in the prior art capable of attaching an elongated cylindrical or rectangular shaped object such as Component C to a flat or curved surface such as those surfaces found on typical surgical retractors.

On the right of the diagram is depicted a large abdominal retractor with a schematic representation of a clip or attachment device attaching the image gathering and light delivery component, Component C of the synergistic system to that retractor. It can be seen from the diagram that the USB cable attaches to the top of this image gathering and light delivery component, Component C of the apparatus.

Surgeons have their favoured preferences for retractors and thus the present disclosure envisages the use of any suitable attachment specie for the purposes of attaching the image gathering and light delivery component, Component C of the apparatus forming the system adapted to fit the many traditional surgical retractors in common use the great number of which are not illustrated nor discussed in the present disclosure, in the interests of brevity.

It will be further appreciated that one advantage of the disclosed adapted apparatus is the facilitation of sterilization and preserving the sterility of the surgical wound zone which is easier to achieve by means of a modular system of different components when some of these are disposable and some not and when there is no physical connection between some of the components employed, in particular between Component A, the Tablet PC or equivalent display component, and Component B, the mini computer dongle.

### Embodiment 2

Attention is directed to FIG. 2 which depicts a cheaper embodiment variation of the disposable image gathering and light delivery component, Component C. This variation provides a means of preserving the CMOS image sensor chip circuit sub-component and LED sub-components to avoid disposing of these by incineration. FIG. 2 depicts a female hood attachment which is capable of attaching a USB cable or equivalent by means of a USB connection or similar connection which would attach to the micro computer dongle Component B to the said hood.

This USB connection and cabling would provide power ground and data bus services to the CMOS image sensor component and LED sub-component located in the hood. The female end of the hood would typically have an appropriately shaped opening which may be cylindrical or rectangular, but not necessarily limited to a cylindrical or a rectangular shaped opening however the said opening would correspond with the shape of the top of Component C such that into which opening the top of Component C would be inserted.

Image gathering and light delivery would take place via the hood at the top of Component. C, where at the top of Component C a Perspex window would make intimate contact with a similar Perspex window just beneath the CMOS sub-component and LED sub-component in the hood. Strands of fibre-optic cables running from the top end of the image gathering light delivery Component C to the bottom close to the wound would respectively convey light photons down so as to illuminate the wound and convey reflected image photons up to the CMOS circuit chip located in the hood. Thus avoiding the need to dispose of the expensive CMOS video camera circuit chips.

The hood could additionally be provided with a flange for the attachment of a disposable sterile plastic sleeve. Alternatively a flange may of course be placed on the disposable image gathering light delivery component C. The plastic sleeve would isolate the hood from body fluids thus obviating the need to dispose of the hood attachment after the operation or procedure.

It will be appreciated that where Component C comprises only a Perspex superstructure or one manufactured from other suitable plastic materials with fibre- optic cables traversing its length this embodiment would be somewhat cheaper to dispose of and more environmentally friendly than a more complex embodiment containing solid-state components.

Further, where the superstructure of Component C is fabricated from plastic material other than Perspex it will be appreciated that one or more windows would be provided at the wound end of Component C, which window(s) would typically be manufactured from transparent Perspex or equivalent material to serve the functionality of a window(s) permitting light photons which had come from the LED and travelled down the strands of fiberoptic cables to exit Component C and illuminate the wound and likewise such a window(s) would also permit reflected photons from the wound to enter Component C and travel upward towards the top of Component C by similar fiberoptic cable strands and traverse the Perspex window at the top of Component C and enter the hood to be received by the CMOS sub-component in the hood. In addition to serving the functionality as a window the Perspex window(s) at the base of Component C may serve the functionality of a lens as discussed herein before.

It will be further appreciated that the plastic outer superstructure of the embodiment just described could be replaced by a stainless steel superstructure in the form of a thick needle having a suitable point at the wound end and an appropriately configured cylindrical male or female head end connection which may be shaped similar to connections for standard needles which connection would attach to a suitably shaped hood which hood having CMOS and LED sub-components and function in a manner as described herein before and which needle shaped Component C would have separate strands of fiberoptic cables traversing its length conveying photons from the hood down to the point and conveying reflected photons up to the CMOS in the hood. At the point end of the said needle shaped Component C would be appropriately configured lens specie sub-components to guide the photons to and from the strands of fibre optic cables.

### Embodiment 3

Attention is drawn to FIG. 3 which depicts an illustration of a more expensive embodiment of Component C of the apparatus forming the system for use in clinically invasive procedures and surgery.

In this embodiment, Component C, comprises its usual sub-components as set forth herein above in Embodiment 1 combined additionally with components normally present in Component B, the micro computer dongle component with its own independent operating system.

In particular this expensive embodiment of Component C comprises:

a Perspex window(s) at the wound end, which may serve the functionality of window and lens; together with

the CMOS image capture chip behind one Perspex window, linked by

a data bus; which databus runs upwards to the processing chips all contained in the same sterile disposable device; the bus provides power and ground and transmits video data upwards by the same bus, alongside the CMOS image sensor chip circuit, video camera chip, or close by lies,

an LED light source similarly powered and grounded by the data bus which serves the CMOS and light emitter component; additionally

the sub-components normally present in component B are also included; in particular,

a CPU chip;

BLUETOOTH® chip circuit and an antenna;

the EPROM chip or ROM or Flash memory chip; holding

the micro operating system or other preferred operating system;

RAM buffer cache(s), and

lithium ion battery for power, or other suitable power source which may alternatively be externally supplied by a jack, not shown, are present in the more expensive embodiment of component C of the adapted apparatus.

The various chips in Embodiment 3 of Component C of the synergistic system wound preferably be linked together by wiring in a suitable way, not shown, which wireing layout being a matter trivial to the skilled artisan skilled in the art of designing wiring layouts for electronic devices. The various chips of Embodiment 3 of Component C of the synergistic system will ultimately be connected to an antenna which will send a stream of video data to Component A, the preferred Tablet PC of the apparatus not shown in this particular diagram.

Typically the more expensive embodiment of Component of C is envisaged to be, but not limited to being, a long rectangular or cylindrical shaped unit measuring 1 cm depth 1 cm and with and perhaps 10 cm in length. However many variations in the dimensions of this embodiment would be readily apparent to the skilled artisan without departing from the scope and spirit of the invention as disclosed.

### Embodiment 4

Attention is drawn to FIG. 4 which depicts a further embodiment of the disclosure for intended use in the field of anaesthesiology. FIG. 4 (a) illustrates the basic problem of difficult intubation where a gum elastic bougie is shown guiding an endotracheal tube (ET tube) past the vocal cords into the trachea.

The endotracheal tube is threaded onto the gum elastic bougie then the gum elastic bougie is inserted through the vocal cords first, after which the ET tube is then railroaded along the bougie into the trachea. Current practice being that the gum elastic bougie is inserted into the vocal cords using direct visualisation using a standard laryngoscope or fibre-optic endoscope when more difficult cases for intubation are anticipated by the anaesthetist or rescue personnel. One embodiment of the present invention is depicted in FIG. 4(b) which provides an illustration of the present invention adapted for use in anaesthesiology.

Diagram FIG. 4(b) shows an embodiment of Component C of the apparatus which Component C takes the form resembling a typical gum elastic bugie which has been modified to become an advanced bougie. Which advanced bougie, resembling an elongated form of Component C comprises additional sub-components. At the laryngeal end of the tip of the modified gum elastic bougie lies a CMOS image sensor chip and close to this a LED light emitting diode or similar component.

Both the CMOS and LED lie behind a suitable Perspex window(s) which window(s) may serve both the functions of window and lens for focus.

Both the CMOS image sensor, video camera circuit chip and the light emitting diode are connected via data bus cabling which provides power and ground and the data bus for image data to be transmitted towards the mouth end of the bougie which provides a USB socket to take the data stream out to Component B and provide the power and ground inward.

This novel advanced bougie in many respects resembles the Component C embodiment depicted in FIG. 1) and as described herein above the difference being it has a greatly elongated shape so it can function also as an anaesthetic bougie as well as an light emitting and image gathering device.

It will be fully appreciated that the high technology bougie may also be configured in accordance with Embodiment 2 if cost savings are desired.

It will be further appreciated that the high technology bougie may be configured in accordance with Embodiment 3 as a more expensive version respectively without departing from the scope and spirit of the invention as disclosed.

It will also be appreciated that the high technology bougie will be connected preferably by suitable USB cable attachments or equivalent (not shown in FIG. 4B but as described elsewhere in the disclosure) to Component B which in turn will communicate preferably by BLUETOOTH® with Component A, a Tablet PC or smartphone as depicted in FIG. 1.

In use the anaesthesiologist would typically guide the high technology bougie through the vocal cords using visualisation obtained from the image displayed on Component A, the Tablet PC or smart-phone or equivalent. He may also use a laryngoscope in addition. Once the bougie has been passed through the vocal cords the USB cable can be temporary disconnected from the bougie and the ET tube can be threaded down along the high technology bougie past the USB connection and then the USB cable would be reconnected so as to permit visualisation of the ET tube vocal chords and trachea on the screen of a smart phone or Tablet PC, Component A, as the ET tube is finally pushed past the vocal cords and into the trachea.

Thus Embodiment 4 provides a high technology bougie which will facilitate the intubation of a patient in difficult situations such as road traffic accident victims at the side of the road or grossly obese patients or those patients with unfavourable necks. It is envisaged that the advanced bougie will be manufactured in several sizes, a normal adult size, a larger adult size, paediatric sizes and micro sizes for intubating neonates.

### METHOD OF INDUSTRIAL USE

The present disclosure further teaches the methods for deploying and using in surgical operating theatres, the apparatus forming the system herein before disclosed. The method(s) for deploying and using the system herinbefore disclosed comprise a number of steps some of which are sequential and some not.

Typical steps comprising the method(s) for using the apparatus are:

In most hospitals operating theatres form part of a suite of theatres and the theatres are assigned codes comprising letters and numbers such as Th/1; Th/2 and the like. Thus it is envisaged that each theatre forming part of a theatre suit would have a set of Component A devices, Tablet PC's, and a set of dongles, Component B. Accordingly it is envisaged that the Tablet PC's and dongles Components A, B respectively would be assigned similar alpha-numeric letter codes such as Th1/1, Th1/2, Th1/3 and the like, and that it would be the duty of the ODA (Operating Department Assistant) or head scrub nurse to ensure that all of the Theatre 1 dongles were paired up with all of the Theatre 1 Tablet PC's. The purpose of this step being to permit hot swapping of dongles and Tablet PC's during lengthy procedures if battery power failed;

It is further envisaged that it would be the duty of the ODA (Operating Department Assistant) or head scrub nurse to ensure that all of the Theatre 1 dongles and Tablet PC's were kept charged and appropriately cleaned and ready for use;

It is envisaged that a typical surgeon may ask for the system to be deployed either before a procedure or during a procedure if visualisation of structures proves difficult. Thus the system should be capable of being deployed without compromising the sterility of an open surgical wound thus necessitating the following further steps:

A cleaned Component A tablet PC is powered up and placed on an appropriate stand;

a cleaned dongle is powered up and attached to an appropriate USB cable which is non-sterile;

the dongle may be tucked away under the sterile drapes close to the anaesthetist or close to the drip cannula between the patients arm and chest or near the ECG leads;

a sterile packet containing the sterile Component C is half opened without touching the inner surface of the outer wrapping layer and the Component C covered by the inner sterile wrapping layer is dropped onto the instrument tray;

a sterile plastic sleeve is similarly dropped onto the instrument tray;

using the appropriate technique the scrub nurse holds open the proximal end of the sleeve to allow passage of the non sterile USB cable which is threaded through and the non scrubbed runner takes the proximal end of the sleeve and attaches this to the dongle, Component B, using tape;

taking care that the non-sterile USB lead never emerges from the distal opening of the sleeve, the scrub nurse advances the top end of Component C into the sleeve and attaches this to the USB cable and then secures the sterile sleeve with appropriate tape around the top end of Component C in a manner similar to preparing Orthopaedic or similar surgical drills;

the image detected by Component C should now display of the Tablet PC and if not connections and devices are checked;
Component C can now be attached by the appropriate attachment specie to the surgeons preferred retractor and used to visualise the wound;
the degree of magnification of images may be adjusted by pinching the screen of Component A using the pinch manoeuvre and preferably this would be executed by a non-scrubbed theatre runner;
during use of the apparatus the surgeon may need to adjust his operating style to accommodate any time lag that may be caused by the electronic circuitry such as the CMOS image sensor chip and the need to transmit the video data stream from the dongle Component B, to the Tablet PC Component A, and to allow for any further delay caused by the Tablet PC processing the said image data stream;
typically, the surgeon would adopt a stroboscopic halting style comprising the sub steps of ACT—STOP—WAIT—ACT—STOP—WAIT.

It will be appreciated that his novel halting style of surgical operating makes use of human neurophysiology and is much easier to adjust to and is less cognitively demanding for the surgeon than adjusting for poor visualisation of structures. By means of the adapted apparatus of the present disclosure the surgeon can harness the powerfull neurocomputation abilities built into his proprioception system which uses the cerebellum. This is so because the adapted apparatus gives the surgeon a clearer indication of where the tip of his instrument is located relative to vital structures. The proprioception system is faster more efficient and more accurate than the visual system. One could sum this up as follows: ‘It is easier and safer to operate with a clearer more accurate view which comes a little late than to operate with a real-time poor quality hazy view’.

1. A novel apparatus adapted for use in clinically invasive proceedures comprising a plurality of objects acting in concert and forming a system, the objectives of which being to display to an operating surgeon or clinician the image of human or animal tissue and illuminate said tissue during the course of surgical or invasive clinical procedures and to improve the reliability of visualisation of the said tissue structures thereby, and comprising:
at least two components which are computers or computer like devices containing microprocessors running independent operating systems; wherein,
Component A, is any commercially available Tablet PC or smart phone or computer device which is capable of displaying high-definition video images and having a touch screen capable of magnifying the said video images to provide a magnified view of the said image; and wherein,
Component A is linked preferably by BLUETOOTH® or other WIFI connection to a second computer or computer like device, Component B; and wherein Component B may be a device similar to that of Component A but is preferably dissimilar; and wherein,
Component B has a separate microprocessor and runs a separate operating system; which operating system may be an operating system system similar to that runing on Component A, but preferably may be a different operating system from that of component A; and wherein,
Component B is linked using cables or other suitable connections to a small sterile disposable component Component C; and wherein
Component C comprises a light emitting element, such as an LED (light emitting diode) or equivalent; and wherein Component C addittionally comprises
a light collecting video imaging chip such as, but not limited to, a CMOS pixel sensor chip or CCD charged coupled device and the like or equivalent; and wherein Component C addittionally comprises,
electro conducting cables traversing its length providing power and ground to the CMOS and LED subcomponents; and wherein Component C addittionally comprises,
a data bus; which databus conveys a video image data stream and control services such as clock signals where required to appropriate connections connecting with Component B and wherein,
Component C is a sterile disposable device; and wherein
Component C is capable of being attached to surgical retractors in current use when required; the entire system being configured so that the apparatus adapted for use in clinically invasive proceedures comprising a plurality of objects acting in concert and forming a system comprising computers and components forms an ensemble specifically designed to be used during surgery and other clinical procedures so as to improve visualisation of structures.

2. A novel apparatus adapted for use in clinically invasive proceedures comprising a plurality of objects acting in concert and forming a system according to claim 1; wherein
Component A of the apparatus, the computer Tablet PC or smart phone, is one of, but is not limited to being, any of the following commercially available Tablet PC's or smart-phone or earlier or later versions thereof, having the capability of displaying high definition video images;
iPad®; iPad mini®; Amazon Kindle Fire HDX7®; Samsung Galaxy Note Pro 12.2®; Microsoft Surface Pro 3®; LG G Pad®; Samsung Galaxy Tab Pro 10. 1®; Google Nexus®; Sony Xperia HTC One®; Nokia Lumia and the like.

3. A novel apparatus adapted for use in clinically invasive proceedures comprising a plurality of objects acting in concert and forming a system according to claim 1; wherein
Component B of the system, an intermediate computer or computer dongle like device, has installed and runs a separate operating system separate from that of component A, the Tablet PC or smart-phone component, which separate operating system running on component B is one of, but is not limited to being, any of the following operating systems;
Android One® ;Android operating systems 4.4® or earlier or later versions thereof such as Ice Cream Sandwich or Android Jelly Bean® or Android KitKat®; Contiki v; Java® based operating systems; Java® based operating systems for embedded devices; Microsoft Internet of Things®; Microsoft Windows 8® or earlier or later versions thereof; Microsoft Windows Phone 7.5® or earlier or later versions thereof; Linux operating systems or versions thereof or or slimmed down version of the Linux kernel; Apple iOS7® or earlier or later versions thereof.

4. A novel apparatus adapted for use in clinically invasive proceedures comprising a plurality of objects acting in concert and forming a system according to claim 1; wherein
Component B of the system, the intermediate computer or computer dongle like device, is attached, by means of an electromagnetically conductive cable, such as but not limited to being, a USB cable, to an intermediate device, preferably hood shaped, but not limited to being hood shaped, which hood shaped intermediate device comprises:
a light emitting diode light source; and
a light gathering CMOS video imaging chip; both housed behind a Perspex window(s); and which hood contains a female opening capable of receiving a male tip from Component C; the sterile light emitting and image gathering component, which male tip comprises,
a corresponding and matching Perspex window(s); so that light from the light emitting diode in the hood can traverse both Perspex windows and enter Component C, and light received from the base of Components C can transverse both Perspex windows and enter the CMOS device located in the hood; and where,
Component C the light emitting light collecting device has strands of fibre-optic cables traversing its length; which fiberoptic cables convey photons from the light emitting diode in the hood to the most inferior aspect of Component C; and
additional fibre-optic cables which collect photons reflected from the wound; and carry said reflected and collected photons upwards towards the Perspex windows and the CMOS device located in the hood attached to the tip of Component C.

5. A novel apparatus adapted for use in clinically invasive proceedures comprising a plurality of objects acting in concert and forming a system according to claim 1; wherein the sub components of component B and the sub components of component C are combined together to form a single device; and wherein,
the intermediate computer components such as the microprocessor; EPROM; RAM memory; the RAM buffer caches; and Bluetooth® chips and antenna; and or battery or other suitable power and ground source normally present in Component B are all contained within the sterile disposable Component C, together with Components normally present in component C, the light emitting, and image sensing CMOS sub-component, and wherein some or all of the said subcomponents are fused onto a single chip.

6. A novel apparatus adapted for use in clinically invasive proceedures comprising a plurality of objects acting in concert and forming a system in accordance with claim 1, 2, 3, and 4, and or 5 wherein,
Component C, the light emitting and image sensing device is a sterilizable disposable specie having the capability of emitting light from its light emitting subcomponent which may be a light emitting diode or other light emitter so as to illuminate tissues and having the capability of receiving reflected photons from tissues and conveying the same so that the image may be convened into a digital data stream.

7. A detachable hood or specie having a light emitter and CMOS image sensing subcomponent in accordance with claim 1, 2, 3, and or 4, and or 5 capable of attaching to Component C, the light emitting and image sensing sterilizable disposable specie.

8. A novel apparatus adapted for use in clinically invasive proceedures comprising a plurality of objects acting in concert and forming a system in accordance with claim 1, 2, 3, 4, 5, 6, 7, wherein Component C, the light emitting and image sensing device takes the form of the gum elastic bougie suitable for use in the field of anaesthesiology to facilitate visualisation of the vocal cords and allow insertion of an endotracheal intubation tube, wherein
the light emitting and image sensing component C visualises the vocal cords and facilitates an endo tracheal tube to be threaded along its length and pass through the vocal cords into the trachea, and wherein, the image obtained by Component C is displayed on a Tablet PC or smart phone, component A, thus permitting the anaesthesiologist to gain a view of the vocal cords by’ means of the image displayed preferably on a tablet PC or smart phone, Component A.

9. A means for illuminating and displaying anatomical structures during surgical or clinical procedures whereby,
the desired image is displayed on a Tablet PC or smart phone screen or
equivalent, in a manner such that the the component displaying the displayed image may be positioned in three dimensions relative to the surgeon so that the surgeon may simultaneously look at the wound directly or may view the displayed images as an indirect view; and whereby
the surgeon may view a magnified view on the display alongside an unmagnified direct view if desired; and whereby
a source of illuminating photons is provided close to the structures desired to be displayed, comprising:
a novel apparatus adapted for use in clinically invasive proceedures comprising a plurality of objects acting in concert and forming a system comprising:
major Component(s) A, B, C, rind additional optional minor components; wherein
Component A is any commercially available tablet PC or smart phone or computer which is capable of displaying high-definition video images, and having a touch screen capable of magnifying the said video images to provide a magnified view; and wherein Component A is linked preferably by Bluetooth® or other WIFI or equivalent signals connection to a second computer or computer like device, Component B; and wherein, Component B may be a device similar to that of Component A, but is preferably smaller and dongle like in form; and wherein,
Component B has a separate microprocessor and a separate operating system, which operating system may be an operating system system similar to that running on component A, but preferably may be a different operating system from that of component A; and wherein, Component B is linked using cables or other suitable connections to a small sterile disposable component, Component C; and wherein, Component C comprises:
a light emitting element, such as an LED (light emitting diode) or equivalent; and
a light collecting video imaging chip such as, but not limited to, a CMOS active pixel sensor or charged coupled device sensor subcomponent or equivalent; and addittionally comprises,
electro conducting cables traversing its length providing power and ground;
and a data bus conveying a data stream and control services such as clock signals when required from Component B, to Component C; and wherein
Component B may be a device similar to that of component A; and wherein Component B has a separate microprocessor and a separate operating system which operating system may be an operating system system similar to that running on component A, but preferably may be a different operating system from that of component A; and wherein Component B is linked using cables or other suitable connections to a small sterile disposable component, Component C; and wherein Component C comprises:
a light emitting element, such as an LED (light emitting diode) and the light collecting video imaging chip such as, but not limited to, a CMOS image sensor chip or a CCD charged coupled device and the like and has electro conducting cables traversing its length providing power and ground and data bus and control services such as clock signals where required from Component B, and wherein Component C is a sterile disposable device; and is capable of being attached to surgical retractors in current use when required; the system being configured so that that the entire synergistic composition of objects comprising computers and components form an ensemble specifically designed to be used during surgery and other clinical procedures so as to improve visualisation of structures.

10. An apparatus adapted for use in clinically invasive proceedures comprising a plurality of objects acting in concert and forming a system according to claims 1, 2, and 9.

11. An apparatus adapted for use in clinically invasive proceedures comprising a plurality of objects acting in concert and forming a system according to claims 1, 3, and 9.

12 An apparatus adapted for use in clinically invasive proceedures comprising a plurality of objects acting in concert and forming a system according to claims 1 ,4 ,and 9.

13 An apparatus adapted for use in clinically invasive proceedures comprising a plurality of objects acting in concert and forming a system according to claims 1, 5, and 9.

14. An apparatus adapted for use in clinically invasive proceedures comprising a plurality of objects acting in concert and forming a system according to claims 1, 6, and 9.

15 An apparatus adapted for use in clinically invasive proceedures comprising a plurality of objects acting in concert and forming a system according to claims 1, 7, and 9.

16. An apparatus adapted for use in clinically invasive proceedures comprising a plurality of objects acting in concert and forming a system according to claims 1, 8, and 9.

17. A method of enabling surgeons:
to see more reliably what they are doing and thereby making surgery easier
and safer to perform; and of improving the illumination of structures in the depths of a wound; and
capturing an image of structures of interest in the said wound; and displaying an image of the said structures to the operating surgeon in a manner without risking undue damage to the said vital structures; and without increasing the risks of contamination by infectious agents; and
permitting the surgeon to view the anatomical structures by easily switch able views magnified and unmagnified, direct and indirect separately or viewed in combination; and
permitting the displayed image to be moved in three dimensional space; and avoiding the need to fix the displayed image in a position directly in front of the wound obscuring the surgeons direct view; and
enhancing the said video images by harnessing the powerful imaging technology of Tablet PC's or smart-phones; and
having a novel apparatus adapted for use in clinically invasive proceedures comprising a plurality of objects acting in concert and forming a system itself comprising:
at least two components which are computers or computer like devices containing microprocessors; and
running independent operating systems; wherein,
Component A, is any commercially available Tablet PC or smart phone or computer device which is capable of displaying high-definition video images, and having a touch screen capable of magnifying the said video images to provide a magnified view of the said image; and wherein,
Component A is linked preferably by BLUETOOTH® or other WIFI connection to a second computer or computer like device Component B; and wherein,
Component B is preferably a dissimilar device to Component A and preferably takes the form of a dongle, but may be similar to Component A; and wherein,
Component B has a separate microprocessor running a separate operating system from Component A; and wherein
Component B may be is non-sterile; and wherein
Component B the dongle is tucked away under the sterile drapes close to the anaesthetist or close to the drip cannula or ECG leads; and
half opening a sterile packet containing the sterile Component C without touching the inner surface of the outer wrapping layer; and
dropping the Component C covered by the inner sterile wrapping layer onto the instrument tray;
dropping a sterile plastic sleeve onto the instrument tray; and
using the appropriate technique the scrub nurse holds open the proximal end of the sleeve to allow passage of the non sterile USB cable, which cable is threaded through the sleeve; and
taking the proximal end of the sleeve by the the non scrubbed runner and who attaches this to the dongle using tape; and
taking care that the non-sterile USB lead never emerges from the distal opening of the sleeve the scrub nurse advances the top end of Component C into the sleeve and attaches up the USB cable and then secures the sterile sleeve with appropriate tape; and
checking that the image detected by Component C is now displayed by the Tablet PC and if not connections are checked; and
the attaching of Component C by the appropriate attachment specie to the surgeons preferred retractor; and
the use of Component A by the surgeon to visualise the wound; and
causing the image of the wound to be magnified by using the pinch manoeuvre on the screen of the Tablet PC; and
adjusting the manner and style of operating such that the surgeon adopts an ‘ACT—STOP—WAIT—ACT—STOP—WAIT’style to adapt to any time lag caused by the electronic circuits.
